(19) United States
(12) Patent Application Publication
(10) Pub. No.: US 2026/0269059 A1
(43) Pub. Date: Sep. 10, 2026

(54) SYSTEM

(71) Applicant: SoftBank Group Corp., Tokyo (JP)

(72) Inventor: Kakushi HARAGUCHI, Tokyo (JP)

(21) Appl. No.: 19/073,215

(22) Filed: Mar. 7, 2025

(51) Int. Cl. G16H 40/67; G16H 20/10; G16H 20/30; G16H 20/60

(52) U.S. Cl. CPC G16H 40/67; G16H 20/10; G16H 20/30; G16H 20/60

(57) ABSTRACT

The system according to the embodiment comprises a reception unit, an AI robot, and a determination unit. The reception unit accepts user registration. The AI robot is lent to the user whose registration has been accepted by the reception unit. The determination unit determines the user's situation based on the information sent from the AI robot.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to a character of a chatbot, encoding the prompt, inputting the encoded prompt into a language model, and generating a chatbot utterance in response to the user utterance.

In conventional technology, effective means for supporting the daily lives of dementia patients and reducing missing persons have not been sufficiently provided, leaving room for improvement.

### SUMMARY OF THE INVENTION

The system according to an embodiment comprises a reception unit, an AI robot, and a determination unit. The reception unit accepts user registration. The AI robot is lent to a user whose registration has been accepted by the reception unit. The determination unit determines a user's situation based on information sent from the AI robot.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment.

FIG. 2 is a conceptual diagram showing an example of main functions of a data processing device and a smart device according to a first embodiment.

FIG. 3 is a conceptual diagram showing an example configuration of a data processing system according to a second embodiment.

FIG. 4 is a conceptual diagram showing an example of main functions of a data processing device and smart glasses according to the second embodiment.

FIG. 5 is a conceptual diagram showing an example configuration of a data processing system according to a third embodiment.

FIG. 6 is a conceptual diagram showing an example of main functions of a data processing device and a headset-type terminal according to the third embodiment.

FIG. 7 is a conceptual diagram showing an example configuration of a data processing system according to a fourth embodiment.

FIG. 8 is a conceptual diagram showing an example of main functions of a data processing device and a robot according to the fourth embodiment.

FIG. 9 shows an emotion map where multiple emotions are mapped.

FIG. 10 shows an emotion map where multiple emotions are mapped.

### DETAILED DESCRIPTION OF THE PREFERRED EMBODIMENTS

Hereinafter, an example of embodiments of the system related to the technology disclosed herein will be described with reference to the attached drawings.

First, the terminology used in the following description will be explained.

In the following embodiments, a processor with a sign (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) with a sign is a memory where information is temporarily stored and used as a work memory by the processor.

In the following embodiments, a storage with a sign is one or more non-volatile storage devices for storing various programs and parameters. Examples of non-volatile storage devices include flash memory (SSD (Solid State Drive) ), magnetic disks (e.g., hard disks), or magnetic tapes, among others.

In the following embodiments, a communication I/F (Interface) with a sign is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

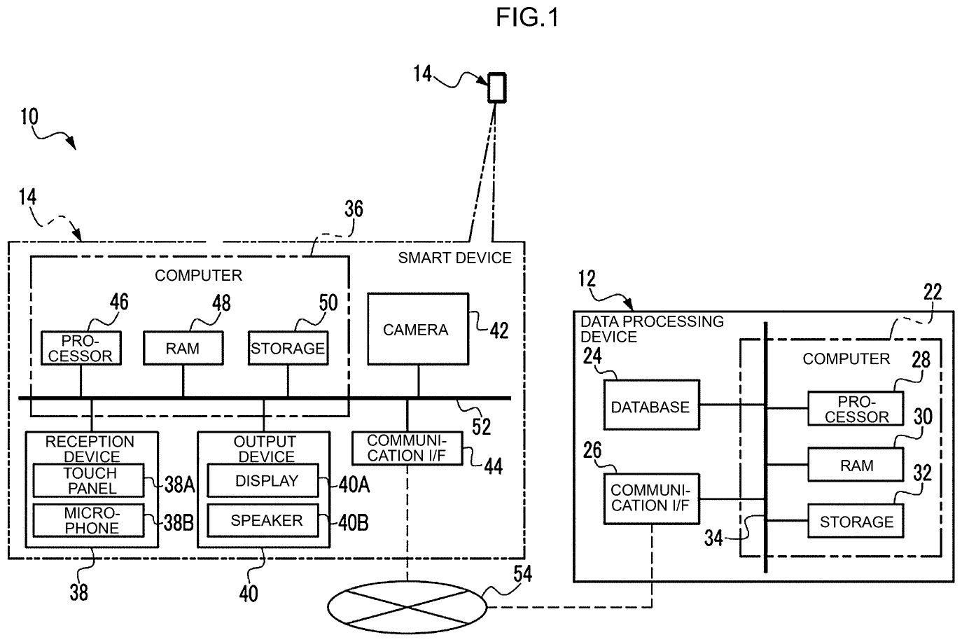

FIG. 1 shows an example configuration of a data processing system 10 according to the first embodiment.

As shown in FIG. 1, the data processing system 10 includes a data processing device 12 and a smart device 14. An example of the data processing device 12 is a server.

The data processing device 12 includes a computer 22, a database 24, and a communication I/F 26. The computer 22 includes a processor 28, a RAM 30, and a storage 32. The processor 28, the RAM 30, and the storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 includes a computer 36, a reception device 38, an output device 40, a camera 42, and a communication I/F 44. The computer 36 includes a processor 46, a RAM 48, and a storage 50. The processor 46, the RAM 48, and the storage 50 are connected to a bus 52. The reception device 38, the output device 40, and the camera 42 are also connected to the bus 52.

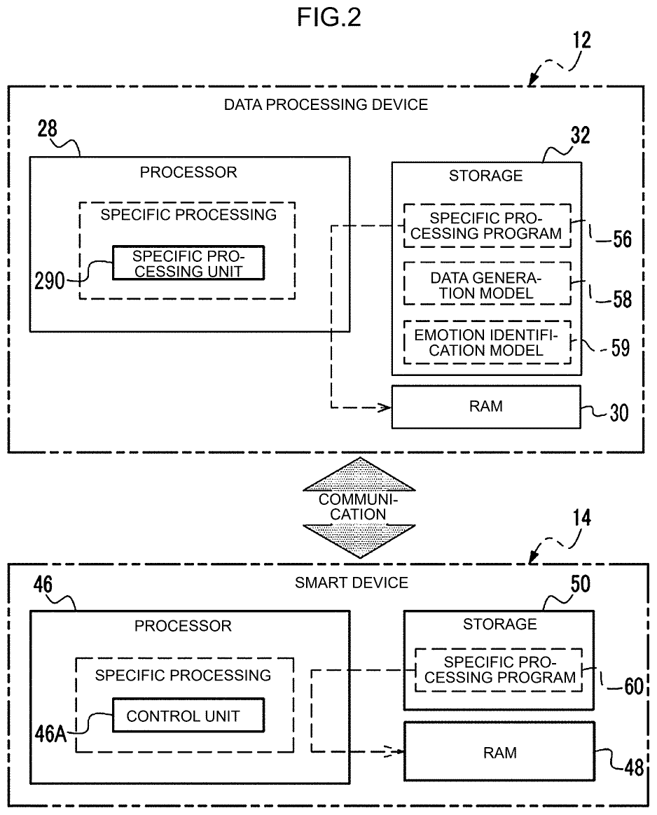

The reception device 38 includes a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 includes a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1 of the Embodiment

The system according to the embodiment aims to support the daily lives of dementia patients and reduce missing persons. This system allows dementia patients to register as users, and AI robots in the form of dogs, cats, or children are lent to the registrants. The AI robot engages in automatic conversation with the user and guides them on times for medication, meals, and exercise. Additionally, the AI robot can remotely monitor the patient's condition (such as health management) and location information. Furthermore, the AI robot collects the user's behavior patterns and conversation content, taking statistics to improve dementia prevention and countermeasures. For example, when a user registers, basic information such as name, address, and contact details is entered into the reception unit. This information is managed by the reception unit, which includes AI processing. Next, the AI robot is lent to the registered user. This AI robot, which takes the form of a dog, cat, or child, engages in automatic conversation with the user. For example, it provides guidance such as “Good morning. It's time to take your medication today.” This conversation is conducted by the conversation unit, which includes AI processing or generative AI processing. Furthermore, the AI robot monitors the user's health management and location information. For example, it detects the user's body temperature and heart rate with sensors and analyzes the data in the determination unit, which includes AI processing. It also tracks the user's location information to help reduce missing persons. This information is managed by the determination unit, which includes AI processing or generative AI processing. Finally, the AI robot collects the user's behavior patterns and conversation content, taking statistics. For example, it records what actions the user takes and what conversations they have, using this information to improve dementia prevention and countermeasures. This information is managed by the output unit, which includes AI processing. This system enables support for the daily lives of dementia patients and is expected to reduce missing persons. Additionally, by monitoring the user's health management and location information, they can live with peace of mind. Furthermore, by collecting behavior patterns and conversation content, dementia prevention and countermeasures can be improved. This enables support for the daily lives of dementia patients and the reduction of missing persons.

The dementia patient support system according to the embodiment includes a reception unit, an AI robot, and a determination unit. The reception unit accepts user registration. User registration includes, for example, online forms or in-person registration. The reception unit inputs the user's basic information (such as name, address, and contact details) and manages it with a system that includes AI processing. For example, the reception unit collects user information through an online form and stores it in a database. In the case of in-person registration, the reception unit manually inputs the user's information and registers it in the system. The AI robot is lent to the registered user. This AI robot, which takes the form of a dog, cat, or child, engages in automatic conversation with the user. For example, the AI robot provides guidance such as “Good morning. It's time to take your medication today.” This conversation is conducted by the conversation unit, which includes AI processing or generative AI processing. The conversation unit uses, for example, speech recognition technology to understand the user's statements and generate appropriate responses. Generative AI uses, for example, text generation AI (such as LLM) to generate natural conversations. Additionally, the conversation unit guides the user on times for medication, meals, and exercise based on the user's statements. For example, the conversation unit provides guidance such as “The next meal time is at 12:00.” The determination unit determines the user's situation based on the information sent from the AI robot. The determination unit detects the user's body temperature and heart rate with sensors and analyzes the data. The determination unit is a system that includes AI processing or generative AI processing, and it monitors the user's health status and location information. For example, the determination unit analyzes the user's body temperature data and issues an alert if there is an abnormality. It also tracks the user's location information to help reduce missing persons. This allows the dementia patient support system according to the embodiment to accept user registration, lend AI robots, and determine the user's situation.

The reception unit accepts user registration. User registration includes, for example, online forms or in-person registration. In the case of online forms, users access the form via the internet and input the necessary information. The form includes detailed information such as name, address, contact details, emergency contact, medical history, and current health status. This information is securely transmitted using security technologies such as SSL encryption and stored in a database. In the case of in-person registration, the reception unit staff manually inputs the user's information. In-person registration is often conducted at hospitals, nursing facilities, or local support centers. The reception unit staff verifies the user's basic information, asks additional questions if necessary, and inputs accurate information into the system. This allows the reception unit to accurately and promptly collect the user's basic information and manage it within the system. Furthermore, the reception unit also has the function to regularly update the user's information. For example, if the user's address changes or there is a change in health status, the user or his/her family can update the information. This ensures that the latest information is always reflected in the system, allowing for appropriate support to be provided.

The AI robot is lent to the registered user. This AI robot, which takes the form of a dog, cat, or child, engages in automatic conversation with the user. The AI robot is designed to support the user's daily life and assist with daily routines. For example, the AI robot starts with a morning greeting and guides the user on times for medication, meals, and exercise. These guides are conducted by the conversation unit, which includes AI processing or generative AI processing. The conversation unit uses speech recognition technology to understand the user's statements and generate appropriate responses.

Generative AI uses text generation AI (such as LLM) to generate natural conversations. For example, if the user asks, “What should I do today?”, the AI robot generates a response such as “Let's take a walk in the morning and have some relaxation time in the afternoon.” Additionally, the AI robot can analyze the user's emotional state and respond appropriately. For example, if the user is feeling anxious, the AI robot offers comforting words such as “It's okay. Is there anything I can help with?” Furthermore, the AI robot collects the user's behavior data and learns daily patterns to provide more personalized support. This allows the AI robot to support the user's daily life and improve the quality of life for dementia patients.

The determination unit determines the user's situation based on the information sent from the AI robot. The determination unit detects the user's body temperature and heart rate with sensors and analyzes the data. The determination unit is a system that includes AI processing or generative AI processing, and it monitors the user's health status and location information. Specifically, sensors installed in the AI robot regularly measure the user's body temperature and heart rate and send the data to the determination unit. The determination unit analyzes these data in real-time and issues an alert if an abnormality is detected. For example, if the user's body temperature rises sharply, the determination unit generates an alert such as “There is a possibility of fever. Please contact a doctor.” Additionally, the determination unit tracks the user's location information to help reduce missing persons. For example, if the user leaves home and does not return for a long time, the determination unit issues an alert such as “The user has left home. Please check.” Furthermore, the determination unit can analyze trends in the user's health status based on past data and conduct long-term risk assessments. This allows the determination unit to continuously monitor the user's health status and support prompt responses by detecting abnormalities early.

The AI robot includes a conversation unit that engages in conversation with the user, a guidance unit that guides the user on times for medication, meals, and exercise, and an output unit that outputs the conversation with the user. The conversation unit engages in conversation with the user. The conversation unit uses, for example, speech recognition technology to understand the user's statements and generate appropriate responses. Generative AI uses, for example, text generation AI (such as LLM) to generate natural conversations. The conversation unit provides guidance such as “Good morning. It's time to take your medication today.” Additionally, the conversation unit guides the user on times for medication, meals, and exercise based on the user's statements. For example, the conversation unit provides guidance such as “The next meal time is at 12:00.” The guidance unit guides the user on times for medication, meals, and exercise. The guidance unit uses, for example, voice guidance or visual guidance to provide information to the user. For example, the guidance unit provides voice guidance such as “The next exercise time is at 15:00.” Additionally, the guidance unit can provide visual guidance using a display. For example, the guidance unit displays the exercise schedule on the display. The output unit outputs the conversation with the user. The output unit uses, for example, voice output or display output to provide information to the user. For example, the output unit greets the user with “Good morning.” in voice. Additionally, the output unit can display the conversation content on the display. For example, the output unit displays “It's time to take your medication today.” on the display. This allows the AI robot to engage in conversation with the user, guide him/her on times for medication, meals, and exercise, and output the conversation.

The determination unit includes a notification unit that notifies the user's family of the determination results. The notification unit provides information to the user's family using, for example, email or SMS. For example, the notification unit notifies the family by email if there is an abnormality in the user's health status. Additionally, the notification unit can provide the user's location information to the family. For example, the notification unit notifies the family by SMS that the user is at a specific location. This allows the determination unit to notify the user's family of the determination results.

The AI robot includes a sensor that detects the user's biometric data, and the output unit outputs the detection results by the sensor in addition to the conversation with the user. The sensor detects the user's biometric data. The sensor acquires data such as heart rate, body temperature, and blood pressure. The sensor monitors the user's health and issues an alert if there is an abnormality. For example, if the user's heart rate is abnormally high, the sensor issues an alert. The output unit provides information to the user using, for example, voice output or display output. For example, the output unit provides guidance such as “Your heart rate is high. Please take a break.” in voice. Additionally, the output unit can display the sensor data on the display. For example, the output unit displays “Body temperature: 37.5 degrees” on the display. This allows the AI robot to detect the user's biometric data and output the results.

The reception unit analyzes the user's past registration history and selects an appropriate registration method. The reception unit analyzes the user's past registration history and selects an appropriate registration method. The reception unit prioritizes proposing registration methods (such as voice or text) that the user has used in the past. Additionally, the reception unit can automatically display frequently entered information as candidates based on the user's past registration history. Furthermore, the reception unit predicts and proposes information to be used at specific times based on the user's past registration history. This allows the reception unit to analyze the user's past registration history and select the optimal registration method.

The reception unit filters data based on the user's current health status and living conditions at the time of registration. The reception unit filters data based on the user's current health status and living conditions at the time of registration. The reception unit proposes appropriate registration options when the user inputs their current health status. Additionally, the reception unit can select the optimal registration method based on the user's living conditions (e. g., living alone or living with family). Furthermore, the reception unit simplifies the registration process according to the user's health status and living conditions. This allows the reception unit to filter data based on the user's current health status and living conditions.

The reception unit prioritizes acquiring highly relevant information by considering the user's geographical location information at the time of registration. The reception unit prioritizes acquiring highly relevant information by considering the user's geographical location information at the time of registration. The reception unit prioritizes acquiring information related to the region if the user lives in a specific area. Additionally, if the user is traveling, the reception unit acquires the optimal information based on the current location. Furthermore, if the user is in a specific facility, the reception unit prioritizes acquiring information related to that facility. This allows the reception unit to prioritize acquiring highly relevant information by considering the user's geographical location information.

The reception unit analyzes the user's social media activity at the time of registration and acquires relevant information. The reception unit analyzes the user's social media activity at the time of registration and acquires relevant information. The reception unit proposes the optimal registration options based on the information shared by the user on social media. Additionally, the reception unit analyzes the user's interests and concerns from their social media activity and acquires relevant information. Furthermore, the reception unit considers the user's social media friendships to acquire relevant information. This allows the reception unit to analyze the user's social media activity and acquire relevant information.

The AI robot analyzes the user's past behavior patterns during the robot's operation and selects appropriate actions. The AI robot analyzes the user's past behavior patterns during the robot's operation and selects appropriate actions. The AI robot selects the optimal actions based on the actions the user preferred in the past. Additionally, the AI robot can perform actions at appropriate times based on the user's past behavior patterns. Furthermore, the AI robot analyzes the user's past behavior patterns to select the most effective actions. This allows the AI robot to analyze the user's past behavior patterns and select the optimal actions.

The AI robot customizes actions based on the user's current living conditions during the robot's operation. The AI robot customizes actions based on the user's current living conditions during the robot's operation. The AI robot selects appropriate actions when the user inputs their current living conditions. Additionally, the AI robot can perform the optimal actions based on the user's living conditions (e.g., living alone or living with family). Furthermore, the AI robot customizes actions according to the user's living conditions. This allows the AI robot to customize actions based on the user's current living conditions.

The determination unit refers to the user's past health data during determination to improve the accuracy of the determination. The determination unit refers to the user's past health data during determination to improve the accuracy of the determination. The determination unit accurately determines the current health status based on the user's past health data. Additionally, the determination unit analyzes specific patterns from the user's past health data to improve the accuracy of the determination. Furthermore, the determination unit refers to the user's past health data to detect abnormalities early. This allows the determination unit to refer to the user's past health data to improve the accuracy of the determination.

The determination unit customizes the determination based on the user's current living conditions during determination. The determination unit customizes the determination based on the user's current living conditions during determination. The determination unit adjusts the determination criteria when the user inputs their current living conditions. Additionally, the determination unit can customize the determination based on the user's living conditions (e.g., living alone or living with family). Furthermore, the determination unit adjusts the determination results according to the user's living conditions. This allows the determination unit to customize the determination based on the user's current living conditions.

The conversation unit refers to the user's past conversation history during conversation to select appropriate conversation content. The conversation unit refers to the user's past conversation history during conversation to select appropriate conversation content. The conversation unit provides related topics based on the content the user has talked about in the past. Additionally, the conversation unit analyzes the user's interests and concerns from their past conversation history to select the optimal conversation content. Furthermore, the conversation unit refers to the user's past conversation history to engage in conversation at appropriate times. This allows the conversation unit to refer to the user's past conversation history to select the optimal conversation content.

The conversation unit customizes the conversation content based on the user's current living conditions during conversation. The conversation unit customizes the conversation content based on the user's current living conditions during conversation. The conversation unit provides appropriate conversation content when the user inputs their current living conditions. Additionally, the conversation unit can select the optimal conversation content based on the user's living conditions (e.g., living alone or living with family). Furthermore, the conversation unit customizes the conversation content according to the user's living conditions. This allows the conversation unit to customize the conversation content based on the user's current living conditions.

The conversation unit determines the priority of the conversation based on the user's submission timing during conversation. The conversation unit determines the priority of the conversation based on the user's submission timing during conversation. The conversation unit prioritizes conversation during specific time slots if the user wishes to converse at those times. Additionally, if the user is in a hurry, the conversation unit engages in conversation quickly. Furthermore, if the user is relaxed, the conversation unit engages in conversation at a leisurely pace. This allows the conversation unit to determine the priority of the conversation based on the user's submission timing.

The conversation unit adjusts the order of the conversation based on the user's relevance during conversation. The conversation unit adjusts the order of the conversation based on the user's relevance during conversation. The conversation unit prioritizes conversation on topics the user shows interest in. Additionally, the conversation unit can structure the conversation in order based on related topics from the user's past conversations. Furthermore, the conversation unit adjusts the order of the conversation based on the user's interests and concerns. This allows the conversation unit to adjust the order of the conversation based on the user's relevance.

The guidance unit refers to the user's past behavior history during guidance to select the optimal guidance content. The guidance unit refers to the user's past behavior history during guidance to select the optimal guidance content. The guidance unit provides related guidance content based on the places the user has visited in the past. Additionally, the guidance unit analyzes the user's interests and concerns from their past behavior history to select the optimal guidance content. Furthermore, the guidance unit refers to the user's past behavior history to provide guidance at appropriate times. This allows the guidance unit to refer to the user's past behavior history to select the optimal guidance content.

The guidance unit customizes the guidance content based on the user's current living conditions during guidance. The guidance unit customizes the guidance content based on the user's current living conditions during guidance. The guidance unit provides appropriate guidance content when the user inputs their current living conditions. Additionally, the guidance unit can select the optimal guidance content based on the user's living conditions (e.g., living alone or living with family). Furthermore, the guidance unit customizes the guidance content according to the user's living conditions. This allows the guidance unit to customize the guidance content based on the user's current living conditions.

The guidance unit determines the priority of the guidance based on the user's submission timing during guidance. The guidance unit determines the priority of the guidance based on the user's submission timing during guidance. The guidance unit prioritizes guidance during specific time slots if the user wishes to receive guidance at those times. Additionally, if the user is in a hurry, the guidance unit provides guidance quickly. Furthermore, if the user is relaxed, the guidance unit provides guidance at a leisurely pace. This allows the guidance unit to determine the priority of the guidance based on the user's submission timing.

The guidance unit adjusts the order of the guidance based on the user's relevance during guidance. The guidance unit adjusts the order of the guidance based on the user's relevance during guidance. The guidance unit prioritizes guidance on topics the user shows interest in. Additionally, the guidance unit can structure the guidance in order based on related topics from the user's past conversations. Furthermore, the guidance unit adjusts the order of the guidance based on the user's interests and concerns. This allows the guidance unit to adjust the order of the guidance based on the user's relevance.

The output unit refers to the user's past data during output to select the optimal output content. The output unit refers to the user's past data during output to select the optimal output content. The output unit provides related output content based on the content the user has talked about in the past. Additionally, the output unit analyzes the user's interests and concerns from their past data to select the optimal output content. Furthermore, the output unit refers to the user's past data to provide output at appropriate times. This allows the output unit to refer to the user's past data to select the optimal output content.

The output unit customizes the output content based on the user's current living conditions during output. The output unit customizes the output content based on the user's current living conditions during output. The output unit provides appropriate output content when the user inputs their current living conditions. Additionally, the output unit can select the optimal output content based on the user's living conditions (e.g., living alone or living with family). Furthermore, the output unit customizes the output content according to the user's living conditions. This allows the output unit to customize the output content based on the user's current living conditions.

The output unit determines the priority of the output based on the user's submission timing during output. The output unit determines the priority of the output based on the user's submission timing during output. The output unit prioritizes output during specific time slots if the user wishes to receive output at those times. Additionally, if the user is in a hurry, the output unit provides output quickly. Furthermore, if the user is relaxed, the output unit provides output at a leisurely pace. This allows the output unit to determine the priority of the output based on the user's submission timing.

The output unit adjusts the order of the output based on the user's relevance during output. The output unit adjusts the order of the output based on the user's relevance during output. The output unit prioritizes output on topics the user shows interest in. Additionally, the output unit can structure the output in order based on related topics from the user's past conversations. Furthermore, the output unit adjusts the order of the output based on the user's interests and concerns. This allows the output unit to adjust the order of the output based on the user's relevance.

The notification unit refers to the user's past data during notification to select the optimal notification content. The notification unit refers to the user's past data during notification to select the optimal notification content. The notification unit provides related notification content based on the notifications the user has received in the past. Additionally, the notification unit analyzes the user's interests and concerns from their past data to select the optimal notification content. Furthermore, the notification unit refers to the user's past data to provide notification at appropriate times. This allows the notification unit to refer to the user's past data to select the optimal notification content.

The notification unit customizes the notification content based on the user's current living conditions during notification. The notification unit customizes the notification content based on the user's current living conditions during notification. The notification unit provides appropriate notification content when the user inputs their current living conditions. Additionally, the notification unit can select the optimal notification content based on the user's living conditions (e.g., living alone or living with family). Furthermore, the notification unit customizes the notification content according to the user's living conditions. This allows the notification unit to customize the notification content based on the user's current living conditions.

The notification unit determines the priority of the notification based on the user's submission timing during notification. The notification unit determines the priority of the notification based on the user's submission timing during notification. The notification unit prioritizes notification during specific time slots if the user wishes to receive notification at those times. Additionally, if the user is in a hurry, the notification unit provides notification quickly. Furthermore, if the user is relaxed, the notification unit provides notification at a leisurely pace. This allows the notification unit to determine the priority of the notification based on the user's submission timing.

The sensor refers to the user's past data during data acquisition to select the optimal acquisition method. The sensor refers to the user's past data during data acquisition to select the optimal acquisition method. The sensor selects the optimal sensor settings based on the user's past data. Additionally, the sensor analyzes specific patterns from the user's past data to select the optimal data acquisition method. Furthermore, the sensor refers to the user's past data to select sensor settings for early detection of abnormalities. This allows the sensor to refer to the user's past data to select the optimal acquisition method.

The sensor considers the user's current living conditions during data acquisition to select the optimal acquisition method. The sensor considers the user's current living conditions during data acquisition to select the optimal acquisition method. Additionally, the sensor can select the optimal data acquisition method based on the user's living conditions (e.g., living alone or living with family). Furthermore, the sensor customizes the sensor's data acquisition method according to the user's living conditions. This allows the sensor to consider the user's current living conditions to select the optimal acquisition method.

The sensor considers the user's geographical location information during data acquisition to select the optimal acquisition method. The sensor considers the user's geographical location information during data acquisition to select the optimal acquisition method. The sensor selects the optimal sensor settings for the region if the user is in a specific area. Additionally, if the user is traveling, the sensor selects the optimal data acquisition method based on the current location. Furthermore, if the user is in a specific facility, the sensor selects the optimal sensor settings for that facility. This allows the sensor to consider the user's geographical location information to select the optimal acquisition method.

The system according to the embodiment is not limited to the examples described above, and various modifications are possible, such as the following.

The AI robot can analyze the user's past behavior patterns and make customized suggestions based on the user's preferences. For example, if the user has exercised at a specific time in the past, the AI robot suggests exercising at that time. Additionally, if the user prefers a specific meal, the AI robot suggests that meal. Furthermore, if the user prefers to visit a specific place, the AI robot suggests visiting that place. This allows the AI robot to make customized suggestions based on the user's past behavior patterns.

The AI robot can customize actions based on the user's current living conditions. For example, if the user lives alone, the AI robot frequently speaks to reduce his/her loneliness. Additionally, if the user lives with family, the AI robot makes suggestions to promote communication with the family. Furthermore, if the user is in a care facility, the AI robot provides guidance according to the facility's schedule. This allows the AI robot to customize actions based on the user's current living conditions.

The determination unit can refer to the user's past health data and detect abnormalities early. For example, the determination unit issues an alert if the current heart rate is abnormally high based on the user's past heart rate data. Additionally, the determination unit issues an alert if the current body temperature is abnormally high based on the user's past body temperature data. Furthermore, the determination unit issues an alert if the current blood pressure is abnormally high based on the user's past blood pressure data. This allows the determination unit to refer to the user's past health data and detect abnormalities early.

The reception unit can analyze the user's past registration history and select an appropriate registration method. For example, the reception unit prioritizes proposing registration methods (such as voice or text) that the user has used in the past. Additionally, the reception unit can automatically display frequently entered information as candidates based on the user's past registration history. Furthermore, the reception unit predicts and proposes information to be used at specific times based on the user's past registration history. This allows the reception unit to analyze the user's past registration history and select the optimal registration method.

The following briefly explains the process flow of Example 1 of the Embodiment.

Step 1: The reception unit accepts user registration. User registration includes online forms or in-person registration, where the user's basic information (such as name, address, and contact details) is input and managed by the system. For example, the reception unit collects user information through an online form and stores it in a database. In the case of in-person registration, the reception unit manually inputs the user's information and registers it in the system.

Step 2: The AI robot is lent to the registered user. This AI robot, which takes the form of a dog, cat, or child, engages in automatic conversation with the user. For example, it provides guidance such as “Good morning. It's time to take your medication today.” The conversation unit uses speech recognition technology to understand the user's statements and generate appropriate responses. Generative AI uses text generation AI (such as LLM) to generate natural conversations. Additionally, the conversation unit guides the user on times for medication, meals, and exercise based on the user's statements.

Step 3: The determination unit determines the user's situation based on the information sent from the AI robot. The determination unit detects the user's body temperature and heart rate with sensors and analyzes the data. The determination unit is a system that includes AI processing or generative AI processing, and it monitors the user's health status and location information. For example, the determination unit analyzes the user's body temperature data and issues an alert if there is an abnormality. It also tracks the user's location information to help reduce missing persons.

### Example 2 of the Embodiment

The system according to the embodiment aims to support the daily lives of dementia patients and reduce missing persons. This system allows dementia patients to register as users, and AI robots in the form of dogs, cats, or children are lent to the registrants. The AI robot engages in automatic conversation with the user and guides them on times for medication, meals, and exercise. Additionally, the AI robot can remotely monitor the patient's condition (such as health management) and location information. Furthermore, the AI robot collects the user's behavior patterns and conversation content, taking statistics to improve dementia prevention and countermeasures. For example, when a user registers, basic information such as name, address, and contact details is entered into the reception unit. This information is managed by the reception unit, which includes AI processing. Next, the AI robot is lent to the registered user. This AI robot, which takes the form of a dog, cat, or child, engages in automatic conversation with the user. For example, it provides guidance such as “Good morning. It's time to take your medication today.” This conversation is conducted by the conversation unit, which includes AI processing or generative AI processing. Furthermore, the AI robot monitors the user's health management and location information. For example, it detects the user's body temperature and heart rate with sensors and analyzes the data in the determination unit, which includes AI processing. It also tracks the user's location information to help reduce missing persons. This information is managed by the determination unit, which includes AI processing or generative AI processing. Finally, the AI robot collects the user's behavior patterns and conversation content, taking statistics to improve dementia prevention and countermeasures. For example, it records what actions the user takes and what conversations they have, using this information to improve dementia prevention and countermeasures. This information is managed by the output unit, which includes AI processing. This system enables support for the daily lives of dementia patients and is expected to reduce missing persons. Additionally, by monitoring the user's health management and location information, they can live with peace of mind. Furthermore, by collecting behavior patterns and conversation content, dementia prevention and countermeasures can be improved. This enables support for the daily lives of dementia patients and the reduction of missing persons.

The dementia patient support system according to the embodiment includes a reception unit, an AI robot, and a determination unit. The reception unit accepts user registration. User registration includes, for example, online forms or in-person registration. The reception unit inputs the user's basic information (such as name, address, and contact details) and manages it with a system that includes AI processing. For example, the reception unit collects user information through an online form and stores it in a database. In the case of in-person registration, the reception unit manually inputs the user's information and registers it in the system. The AI robot is lent to the registered user. This AI robot, which takes the form of a dog, cat, or child, engages in automatic conversation with the user. For example, the AI robot provides guidance such as “Good morning. It's time to take your medication today.” This conversation is conducted by the conversation unit, which includes AI processing or generative AI processing. The conversation unit uses, for example, speech recognition technology to understand the user's statements and generate appropriate responses. Generative AI uses, for example, text generation AI (such as LLM) to generate natural conversations. Additionally, the conversation unit guides the user on times for medication, meals, and exercise based on the user's statements. For example, the conversation unit provides guidance such as “The next meal time is at 12:00.” The determination unit determines the user's situation based on the information sent from the AI robot. The determination unit detects the user's body temperature and heart rate with sensors and analyzes the data. The determination unit is a system that includes AI processing or generative AI processing, and it monitors the user's health status and location information. For example, the determination unit analyzes the user's body temperature data and issues an alert if there is an abnormality. It also tracks the user's location information to help reduce missing persons. This allows the dementia patient support system according to the embodiment to accept user registration, lend AI robots, and determine the user's situation.

The reception unit accepts user registration. User registration includes, for example, online forms or in-person registration. In the case of online forms, users access the form via the internet and input the necessary information. The form includes detailed information such as name, address, contact details, emergency contact, medical history, and current health status. This information is securely transmitted using security technologies such as SSL encryption and stored in a database. In the case of in-person registration, the reception unit staff manually inputs the user's information. In-person registration is often conducted at hospitals, nursing facilities, or local support centers. The reception unit staff verifies the user's basic information, asks additional questions if necessary, and inputs accurate information into the system. This allows the reception unit to accurately and promptly collect the user's basic information and manage it within the system. Furthermore, the reception unit also has the function to regularly update the user's information. For example, if the user's address changes or there is a change in health status, the user or his/her family can update the information. This ensures that the latest information is always reflected on the system, allowing for appropriate support to be provided.

The AI robot is lent to the registered user. This AI robot, which takes the form of a dog, cat, or child, engages in automatic conversation with the user. The AI robot is designed to support the user's daily life and assist with daily routines. For example, the AI robot starts with a morning greeting and guides the user on times for medication, meals, and exercise. These guides are conducted by the conversation unit, which includes AI processing or generative AI processing. The conversation unit uses speech recognition technology to understand the user's statements and generate appropriate responses. Generative AI uses text generation AI (such as LLM) to generate natural conversations. For example, if the user asks, “What should I do today?”, the AI robot generates a response such as “Let's take a walk in the morning and have some relaxation time in the afternoon.” Additionally, the AI robot can analyze the user's emotional state and respond appropriately. For example, if the user is feeling anxious, the AI robot offers comforting words such as “It's okay. Is there anything I can help with?” Furthermore, the AI robot collects the user's behavior data and learns daily patterns to provide more personalized support. This allows the AI robot to support the user's daily life and improve the quality of life for dementia patients.

The determination unit determines the user's situation based on the information sent from the AI robot. The determination unit detects the user's body temperature and heart rate with sensors and analyzes the data. The determination unit is a system that includes AI processing or generative AI processing, and it monitors the user's health status and location information. Specifically, sensors installed in the AI robot regularly measure the user's body temperature and heart rate and send the data to the determination unit. The determination unit analyzes these data in real-time and issues an alert if an abnormality is detected. For example, if the user's body temperature rises sharply, the determination unit generates an alert such as “There is a possibility of fever. Please contact a doctor.” Additionally, the determination unit tracks the user's location information to help reduce missing persons. For example, if the user leaves home and does not return for a long time, the determination unit issues an alert such as “The user has left home. Please check.” Furthermore, the determination unit can analyze trends in the user's health status based on past data and conduct long-term risk assessments. This allows the determination unit to continuously monitor the user's health status and support prompt responses by detecting abnormalities early.

The AI robot includes a conversation unit that engages in conversation with the user, a guidance unit that guides the user on times for medication, meals, and exercise, and an output unit that outputs the conversation with the user. The conversation unit engages in conversation with the user. The conversation unit uses, for example, speech recognition technology to understand the user's statements and generate appropriate responses. Generative AI uses, for example, text generation AI (such as LLM) to generate natural conversations. The conversation unit provides guidance such as “Good morning. It's time to take your medication today.” Additionally, the conversation unit guides the user on times for medication, meals, and exercise based on the user's statements. For example, the conversation unit provides guidance such as “The next meal time is at 12:00.” The guidance unit guides the user on times for medication, meals, and exercise. The guidance unit uses, for example, voice guidance or visual guidance to provide information to the user. For example, the guidance unit provides voice guidance such as “The next exercise time is at 15:00.” Additionally, the guidance unit can provide visual guidance using a display. For example, the guidance unit displays the exercise schedule on the display. The output unit outputs the conversation with the user. The output unit uses, for example, voice output or display output to provide information to the user. For example, the output unit greets the user with “Good morning.” in voice. Additionally, the output unit can display the conversation content on the display. For example, the output unit displays “It's time to take your medication today.” on the display. This allows the AI robot to engage in conversation with the user, guide them on times for medication, meals, and exercise, and output the conversation.

The determination unit includes a notification unit that notifies the user's family of the determination results. The notification unit provides information to the user's family using, for example, email or SMS. For example, the notification unit notifies the family by email if there is an abnormality in the user's health status. Additionally, the notification unit can provide the user's location information to the family. For example, the notification unit notifies the family by SMS that the user is at a specific location. This allows the determination unit to notify the user's family of the determination results.

The AI robot includes a sensor that detects the user's biometric data, and the output unit outputs the detection results by the sensor in addition to the conversation with the user. The sensor detects the user's biometric data. The sensor acquires data such as heart rate, body temperature, and blood pressure. The sensor monitors the user's health and issues an alert if there is an abnormality. For example, if the user's heart rate is abnormally high, the sensor issues an alert. The output unit provides information to the user using, for example, voice output or display output. For example, the output unit provides guidance such as “Your heart rate is high. Please take a break.” in voice. Additionally, the output unit can display the sensor data on the display. For example, the output unit displays “Body temperature: 37.5 degrees” on the display. This allows the AI robot to detect the user's biometric data and output the results.

The reception unit estimates the user's emotions and simplifies the registration process based on the estimated emotions. The reception unit estimates the user's emotions and simplifies the registration process based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the reception unit provides a simple and intuitive interface to quickly complete the registration process. Additionally, if the user is relaxed, the reception unit provides an interface with detailed explanations to carefully complete the registration process. Furthermore, if the user is in a hurry, the reception unit prioritizes voice input to quickly complete the registration process. This allows the reception unit to simplify the registration process based on the user's emotions.

The reception unit analyzes the user's past registration history and selects an appropriate registration method. The reception unit analyzes the user's past registration history and selects an appropriate registration method. The reception unit prioritizes proposing registration methods (such as voice or text) that the user has used in the past. Additionally, the reception unit can automatically display frequently entered information as candidates based on the user's past registration history. Furthermore, the reception unit predicts and proposes information to be used at specific times based on the user's past registration history. This allows the reception unit to analyze the user's past registration history and select the optimal registration method.

The reception unit filters data based on the user's current health status and living conditions at the time of registration. The reception unit filters data based on the user's current health status and living conditions at the time of registration. The reception unit proposes appropriate registration options when the user inputs their current health status. Additionally, the reception unit can select the optimal registration method based on the user's living conditions (e.g., living alone or living with family). Furthermore, the reception unit simplifies the registration process according to the user's health status and living conditions. This allows the reception unit to filter data based on the user's current health status and living conditions.

The reception unit estimates the user's emotions and determines the priority of registration information based on the estimated emotions. The reception unit estimates the user's emotions and determines the priority of registration information based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling stressed, the reception unit prioritizes the input of important information. Additionally, if the user is relaxed, the reception unit allows the input of detailed information. Furthermore, if the user is in a hurry, the reception unit prioritizes the input of minimal information. This allows the reception unit to determine the priority of registration information based on the user's emotions.

The reception unit prioritizes acquiring highly relevant information by considering the user's geographical location. information at the time of registration. The reception unit prioritizes acquiring highly relevant information by considering the user's geographical location information at the time of registration. The reception unit prioritizes acquiring information related to the region if the user lives in a specific area. Additionally, if the user is traveling, the reception unit acquires the optimal information based on the current location. Furthermore, if the user is in a specific facility, the reception unit prioritizes acquiring information related to that facility. This allows the reception unit to prioritize acquiring highly relevant information by considering the user's geographical location information.

The reception unit analyzes the user's social media activity at the time of registration and acquires relevant information. The reception unit analyzes the user's social media activity at the time of registration and acquires relevant information. The reception unit proposes the optimal registration options based on the information shared by the user on social media. Additionally, the reception unit analyzes the user's interests and concerns from their social media activity and acquires relevant information. Furthermore, the reception unit considers the user's social media friendships to acquire relevant information. This allows the reception unit to analyze the user's social media activity and acquire relevant information.

The AI robot estimates the user's emotions and adjusts the robot's actions and conversation content based on the estimated emotions. The AI robot estimates the user's emotions and adjusts the robot's actions and conversation content based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the AI robot speaks in a gentle voice. Additionally, if the user is relaxed, the AI robot provides enjoyable topics. Furthermore, if the user is excited, the AI robot engages in conversation to calm them down. This allows the AI robot to adjust the robot's actions and conversation content based on the user's emotions.

The AI robot analyzes the user's past behavior patterns during the robot's operation and selects appropriate actions. The AI robot analyzes the user's past behavior patterns during the robot's operation and selects appropriate actions. The AI robot selects the optimal actions based on the actions the user preferred in the past. Additionally, the AI robot can perform actions at appropriate times based on the user's past behavior patterns. Furthermore, the AI robot analyzes the user's past behavior patterns to select the most effective actions. This allows the AI robot to analyze the user's past behavior patterns and select the optimal actions.

The AI robot customizes actions based on the user's current living conditions during the robot's operation. The AI robot customizes actions based on the user's current living conditions during the robot's operation. The AI robot selects appropriate actions when the user inputs their current living conditions. Additionally, the AI robot can perform the optimal actions based on the user's living conditions (e.g., living alone or living with family). Furthermore, the AI robot customizes actions according to the user's living conditions. This allows the AI robot to customize actions based on the user's current living conditions.

The determination unit estimates the user's emotions and adjusts the determination criteria based on the estimated emotions. The determination unit estimates the user's emotions and adjusts the determination criteria based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the determination unit relaxes the determination criteria to provide reassurance. Additionally, if the user is relaxed, the determination unit applies strict criteria for accurate determination. Furthermore, if the user is excited, the determination unit adjusts the criteria to encourage calm judgment. This allows the determination unit to adjust the determination criteria based on the user's emotions.

The determination unit refers to the user's past health data during determination to improve the accuracy of the determination. The determination unit refers to the user's past health data during determination to improve the accuracy of the determination. The determination unit accurately determines the current health status based on the user's past health data. Additionally, the determination unit analyzes specific patterns from the user's past health data to improve the accuracy of the determination. Furthermore, the determination unit refers to the user's past health data to detect abnormalities early. This allows the determination unit to refer to the user's past health data to improve the accuracy of the determination.

The determination unit customizes the determination based on the user's current living conditions during determination. The determination unit customizes the determination based on the user's current living conditions during determination. The determination unit adjusts the determination criteria when the user inputs their current living conditions. Additionally, the determination unit can customize the determination based on the user's living conditions (e.g., living alone or living with family). Furthermore, the determination unit adjusts the determination results according to the user's living conditions. This allows the determination unit to customize the determination based on the user's current living conditions.

The conversation unit estimates the user's emotions and adjusts the expression method of the conversation based on the estimated emotions. The conversation unit estimates the user's emotions and adjusts the expression method of the conversation based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the conversation unit engages in conversation using gentle language. Additionally, if the user is relaxed, the conversation unit engages in conversation using friendly language. Furthermore, if the user is excited, the conversation unit engages in conversation using calming language. This allows the conversation unit to adjust the expression method of the conversation based on the user's emotions.

The conversation unit refers to the user's past conversation history during conversation to select appropriate conversation content. The conversation unit refers to the user's past conversation history during conversation to select appropriate conversation content. The conversation unit provides related topics based on the content the user has talked about in the past. Additionally, the conversation unit analyzes the user's interests and concerns from their past conversation history to select the optimal conversation content. Furthermore, the conversation unit refers to the user's past conversation history to engage in conversation at appropriate times. This allows the conversation unit to refer to the user's past conversation history to select the optimal conversation content.

The conversation unit customizes the conversation content based on the user's current living conditions during conversation. The conversation unit customizes the conversation content based on the user's current living conditions during conversation. The conversation unit provides appropriate conversation content when the user inputs their current living conditions. Additionally, the conversation unit can select the optimal conversation content based on the user's living conditions (e.g., living alone or living with family). Furthermore, the conversation unit customizes the conversation content according to the user's living conditions. This allows the conversation unit to customize the conversation content based on the user's current living conditions.

The conversation unit estimates the user's emotions and adjusts the length of the conversation based on the estimated emotions. The conversation unit estimates the user's emotions and adjusts the length of the conversation based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is in a hurry, the conversation unit engages in short and concise conversation. Additionally, if the user is relaxed, the conversation unit engages in longer conversation with detailed explanations. Furthermore, if the user is excited, the conversation unit engages in short conversation to calm him/her down. This allows the conversation unit to adjust the length of the conversation based on the user's emotions.

The conversation unit determines the priority of the conversation based on the user's submission timing during conversation. The conversation unit determines the priority of the conversation based on the user's submission timing during conversation. The conversation unit prioritizes conversation during specific time slots if the user wishes to converse at those times. Additionally, if the user is in a hurry, the conversation unit engages in conversation quickly. Furthermore, if the user is relaxed, the conversation unit engages in conversation at a leisurely pace. This allows the conversation unit to determine the priority of the conversation based on the user's submission timing.

The conversation unit adjusts the order of the conversation based on the user's relevance during conversation. The conversation unit adjusts the order of the conversation based on the user's relevance during conversation. The conversation unit prioritizes conversation on topics the user shows interest in. Additionally, the conversation unit can structure the conversation in order based on related topics from the user's past conversations. Furthermore, the conversation unit adjusts the order of the conversation based on the user's interests and concerns. This allows the conversation unit to adjust the order of the conversation based on the user's relevance.

The guidance unit estimates the user's emotions and adjusts the expression method of the guidance based on the estimated emotions. The guidance unit estimates the user's emotions and adjusts the expression method of the guidance based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the guidance unit provides guidance using gentle language. Additionally, if the user is relaxed, the guidance unit provides guidance using friendly language. Furthermore, if the user is excited, the guidance unit provides guidance using calming language. This allows the guidance unit to adjust the expression method of the guidance based on the user's emotions.

The guidance unit refers to the user's past behavior history during guidance to select the optimal guidance content. The guidance unit refers to the user's past behavior history during guidance to select the optimal guidance content. The guidance unit provides related guidance content based on the places the user has visited in the past. Additionally, the guidance unit analyzes the user's interests and concerns from their past behavior history to select the optimal guidance content. Furthermore, the guidance unit refers to the user's past behavior history to provide guidance at appropriate times. This allows the guidance unit to refer to the user's past behavior history to select the optimal guidance content.

The guidance unit customizes the guidance content based on the user's current living conditions during guidance. The guidance unit customizes the guidance content based on the user's current living conditions during guidance. The guidance unit provides appropriate guidance content when the user inputs their current living conditions. Additionally, the guidance unit can select the optimal guidance content based on the user's living conditions (e.g., living alone or living with family). Furthermore, the guidance unit customizes the guidance content according to the user's living conditions. This allows the guidance unit to customize the guidance content based on the user's current living conditions.

The guidance unit estimates the user's emotions and adjusts the length of the guidance based on the estimated emotions. The guidance unit estimates the user's emotions and adjusts the length of the guidance based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is in a hurry, the guidance unit provides short and concise guidance. Additionally, if the user is relaxed, the guidance unit provides longer guidance with detailed explanations. Furthermore, if the user is excited, the guidance unit provides short guidance to calm them down. This allows the guidance unit to adjust the length of the guidance based on the user's emotions.

The guidance unit determines the priority of the guidance based on the user's submission timing during guidance. The guidance unit determines the priority of the guidance based on the user's submission timing during guidance. The guidance unit prioritizes guidance during specific time slots if the user wishes to receive guidance at those times. Additionally, if the user is in a hurry, the guidance unit provides guidance quickly. Furthermore, if the user is relaxed, the guidance unit provides guidance at a leisurely pace. This allows the guidance unit to determine the priority of the guidance based on the user's submission timing.

The guidance unit adjusts the order of the guidance based on the user's relevance during guidance. The guidance unit adjusts the order of the guidance based on the user's relevance during guidance. The guidance unit prioritizes guidance on topics the user shows interest in. Additionally, the guidance unit can structure the guidance in order based on related topics from the user's past conversations. Furthermore, the guidance unit adjusts the order of the guidance based on the user's interests and concerns. This allows the guidance unit to adjust the order of the guidance based on the user's relevance.

The output unit estimates the user's emotions and adjusts the expression method of the output based on the estimated emotions. The output unit estimates the user's emotions and adjusts the expression method of the output based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the output unit provides output using gentle language. Additionally, if the user is relaxed, the output unit provides output using friendly language. Furthermore, if the user is excited, the output unit provides output using calming language. This allows the output unit to adjust the expression method of the output based on the user's emotions.

The output unit refers to the user's past data during output to select the optimal output content. The output unit refers to the user's past data during output to select the optimal output content. The output unit provides related output content based on the content the user has talked about in the past. Additionally, the output unit analyzes the user's interests and concerns from their past data to select the optimal output content. Furthermore, the output unit refers to the user's past data to provide output at appropriate times. This allows the output unit to refer to the user's past data to select the optimal output content.

The output unit customizes the output content based on the user's current living conditions during output. The output unit customizes the output content based on the user's current living conditions during output. The output unit provides appropriate output content when the user inputs their current living conditions. Additionally, the output unit can select the optimal output content based on the user's living conditions (e.g., living alone or living with family). Furthermore, the output unit customizes the output content according to the user's living conditions. This allows the output unit to customize the output content based on the user's current living conditions.

The output unit estimates the user's emotions and adjusts the length of the output based on the estimated emotions. The output unit estimates the user's emotions and adjusts the length of the output based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is in a hurry, the output unit provides short and concise output. Additionally, if the user is relaxed, the output unit provides longer output with detailed explanations. Furthermore, if the user is excited, the output unit provides short output to calm them down. This allows the output unit to adjust the length of the output based on the user's emotions.

The output unit determines the priority of the output based on the user's submission timing during output. The output unit determines the priority of the output based on the user's submission timing during output. The output unit prioritizes output during specific time slots if the user wishes to receive output at those times. Additionally, if the user is in a hurry, the output unit provides output quickly. Furthermore, if the user is relaxed, the output unit provides output at a leisurely pace. This allows the output unit to determine the priority of the output based on the user's submission timing.

The output unit adjusts the order of the output based on the user's relevance during output. The output unit adjusts the order of the output based on the user's relevance during output. The output unit prioritizes output on topics the user shows interest in. Additionally, the output unit can structure the output in order based on related topics from the user's past conversations. Furthermore, the output unit adjusts the order of the output based on the user's interests and concerns. This allows the output unit to adjust the order of the output based on the user's relevance.

The notification unit estimates the user's emotions and adjusts the expression method of the notification based on the estimated emotions. The notification unit estimates the user's emotions and adjusts the expression method of the notification based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the notification unit provides notification using gentle language. Additionally, if the user is relaxed, the notification unit provides notification using friendly language. Furthermore, if the user is excited, the notification unit provides notification using calming language. This allows the notification unit to adjust the expression method of the notification based on the user's emotions.

The notification unit refers to the user's past data during notification to select the optimal notification content. The notification unit refers to the user's past data during notification to select the optimal notification content. The notification unit provides related notification content based on the notifications the user has received in the past. Additionally, the notification unit analyzes the user's interests and concerns from their past data to select the optimal notification content. Furthermore, the notification unit refers to the user's past data to provide notification at appropriate times. This allows the notification unit to refer to the user's past data to select the optimal notification content.

The notification unit customizes the notification content based on the user's current living conditions during notification. The notification unit customizes the notification content based on the user's current living conditions during notification. The notification unit provides appropriate notification content when the user inputs their current living conditions. Additionally, the notification unit can select the optimal notification content based on the user's living conditions (e.g., living alone or living with family). Furthermore, the notification unit customizes the notification content according to the user's living conditions. This allows the notification unit to customize the notification content based on the user's current living conditions.

The notification unit estimates the user's emotions and adjusts the length of the notification based on the estimated emotions. The notification unit estimates the user's emotions and adjusts the length of the notification based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is in a hurry, the notification unit provides short and concise notification. Additionally, if the user is relaxed, the notification unit provides longer notification with detailed explanations. Furthermore, if the user is excited, the notification unit provides short notification to calm them down. This allows the notification unit to adjust the length of the notification based on the user's emotions.

The notification unit determines the priority of the notification based on the user's submission timing during notification. The notification unit determines the priority of the notification based on the user's submission timing during notification. The notification unit prioritizes notification during specific time slots if the user wishes to receive notification at those times. Additionally, if the user is in a hurry, the notification unit provides notification quickly. Furthermore, if the user is relaxed, the notification unit provides notification at a leisurely pace. This allows the notification unit to determine the priority of the notification based on the user's submission timing.

The sensor estimates the user's emotions and adjusts the sensitivity of the sensor based on the estimated emotions. The sensor estimates the user's emotions and adjusts the sensitivity of the sensor based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the sensor increases sensitivity to acquire detailed data. Additionally, if the user is relaxed, the sensor sets sensitivity to normal. Furthermore, if the user is excited, the sensor adjusts sensitivity to acquire stable data. This allows the sensor to adjust the sensitivity of the sensor based on the user's emotions.

The sensor refers to the user's past data during data acquisition to select the optimal acquisition method. The sensor refers to the user's past data during data acquisition to select the optimal acquisition method. The sensor selects the optimal sensor settings based on the user's past data. Additionally, the sensor analyzes specific patterns from the user's past data to select the optimal data acquisition method. Furthermore, the sensor refers to the user's past data to select sensor settings for early detection of abnormalities. This allows the sensor to refer to the user's past data to select the optimal acquisition method.

The sensor considers the user's current living conditions during data acquisition to select the optimal acquisition method. The sensor considers the user's current living conditions during data acquisition to select the optimal acquisition method. The sensor selects the optimal sensor settings when the user inputs their current living conditions. Additionally, the sensor can select the optimal data acquisition method based on the user's living conditions (e.g., living alone or living with family). Furthermore, the sensor customizes the sensor's data acquisition method according to the user's living conditions. This allows the sensor to consider the user's current living conditions to select the optimal acquisition method.

The sensor estimates the user's emotions and adjusts the frequency of data acquisition based on the estimated emotions. The sensor estimates the user's emotions and adjusts the frequency of data acquisition based on the estimated emotions. Emotion estimation is realized using, for example, an emotion engine or generative AI with emotion estimation functions. Generative AI includes text generation AI (such as LLM) or multimodal generative AI, but is not limited to these examples. For example, if the user is feeling anxious, the sensor increases the frequency of data acquisition to acquire detailed data. Additionally, if the user is relaxed, the sensor sets the frequency of data acquisition to normal. Furthermore, if the user is excited, the sensor adjusts the frequency of data acquisition to acquire stable data. This allows the sensor to adjust the frequency of data acquisition based on the user's emotions.

The sensor considers the user's geographical location information during data acquisition to select the optimal acquisition method. The sensor considers the user's geographical location information during data acquisition to select the optimal acquisition method. The sensor selects the optimal sensor settings for the region if the user is in a specific area. Additionally, if the user is traveling, the sensor selects the optimal data acquisition method based on the current location. Furthermore, if the user is in a specific facility, the sensor selects the optimal sensor settings for that facility. This allows the sensor to consider the user's geographical location information to select the optimal acquisition method.

The system according to the embodiment is not limited to the examples described above, and various modifications are possible, such as the following.

The AI robot can estimate the user's emotions and predict the user's behavior based on the estimated emotions. For example, if the user is feeling anxious, the AI robot predicts that the user is likely to refrain from going out and suggests indoor activities. Additionally, if the user is relaxed, the AI robot predicts that the user is likely to go for a walk and guides the walk time. Furthermore, if the user is excited, the AI robot predicts that the user is likely to exercise and guides the exercise time. This allows the AI robot to predict behavior based on the user's emotions and provide appropriate guidance.

The AI robot can analyze the user's past behavior patterns and make customized suggestions based on the user's preferences. For example, if the user has exercised at a specific time in the past, the AI robot suggests exercising at that time. Additionally, if the user prefers a specific meal, the AI robot suggests that meal. Furthermore, if the user prefers to visit a specific place, the AI robot suggests visiting that place. This allows the AI robot to make customized suggestions based on the user's past behavior patterns.

The determination unit can estimate the user's emotions and detect abnormalities in health status early based on the estimated emotions. For example, if the user is feeling anxious, the determination unit judges that the stress level is high and issues an alert for stress reduction. Additionally, if the user is relaxed, the determination unit judges that the health status is stable and does not issue a special alert. Furthermore, if the user is excited, the determination unit detects abnormalities in heart rate or blood pressure and issues an alert to prompt appropriate action. This allows the determination unit to detect abnormalities in health status early based on the user's emotions.

The AI robot can customize actions based on the user's current living conditions. For example, if the user lives alone, the AI robot frequently speaks to reduce loneliness. Additionally, if the user lives with family, the AI robot makes suggestions to promote communication with the family. Furthermore, if the user is in a care facility, the AI robot provides guidance according to the facility's schedule. This allows the AI robot to customize actions based on the user's current living conditions.

The reception unit can estimate the user's emotions and simplify the registration process based on the estimated emotions. For example, if the user is feeling anxious, the reception unit provides a simple and intuitive interface to quickly complete the registration process. Additionally, if the user is relaxed, the reception unit provides an interface with detailed explanations to carefully complete the registration process. Furthermore, if the user is in a hurry, the reception unit prioritizes voice input to quickly complete the registration process. This allows the reception unit to simplify the registration process based on the user's emotions.

The AI robot can estimate the user's emotions and manage the user's stress level based on the estimated emotions. For example, if the user is feeling anxious, the AI robot plays music to relax. Additionally, if the user is relaxed, the AI robot suggests activities to maintain that state. Furthermore, if the user is excited, the AI robot guides deep breathing or meditation. This allows the AI robot to manage the user's stress level based on the user's emotions.

The determination unit can refer to the user's past health data and detect abnormalities early. For example, the determination unit issues an alert if the current heart rate is abnormally high based on the user's past heart rate data. Additionally, the determination unit issues an alert if the current body temperature is abnormally high based on the user's past body temperature data. Furthermore, the determination unit issues an alert if the current blood pressure is abnormally high based on the user's past blood pressure data. This allows the determination unit to refer to the user's past health data and detect abnormalities early.

The AI robot can estimate the user's emotions and predict the user's behavior based on the estimated emotions. For example, if the user is feeling anxious, the AI robot predicts that the user is likely to refrain from going out and suggests indoor activities. Additionally, if the user is relaxed, the AI robot predicts that the user is likely to go for a walk and guides the walk time. Furthermore, if the user is excited, the AI robot predicts that the user is likely to exercise and guides the exercise time. This allows the AI robot to predict behavior based on the user's emotions and provide appropriate guidance.

The reception unit can analyze the user's past registration history and select an appropriate registration method. For example, the reception unit prioritizes proposing registration methods (such as voice or text) that the user has used in the past. Additionally, the reception unit can automatically display frequently entered information as candidates based on the user's past registration history. Furthermore, the reception unit predicts and proposes information to be used at specific times based on the user's past registration history. This allows the reception unit to analyze the user's past registration history and select the optimal registration method.

The AI robot can estimate the user's emotions and adjust the robot's actions and conversation content based on the estimated emotions. For example, if the user is feeling anxious, the AI robot speaks in a gentle voice. Additionally, if the user is relaxed, the AI robot provides enjoyable topics. Furthermore, if the user is excited, the AI robot engages in conversation to calm them down. This allows the AI robot to adjust the robot's actions and conversation content based on the user's emotions.

The following briefly explains the process flow of Example 2 of the Embodiment.

Step 1: The reception unit accepts user registration. User registration includes online forms or in-person registration, where the user's basic information (such as name, address, and contact details) is input and managed by the system. For example, the reception unit collects user information through an online form and stores it in a database. In the case of in-person registration, the reception unit manually inputs the user's information and registers it in the system.

Step 2: The AI robot is lent to the registered user. This AI robot, which takes the form of a dog, cat, or child, engages in automatic conversation with the user. For example, it provides guidance such as “Good morning. It's time to take your medication today.” The conversation unit uses speech recognition technology to understand the user's statements and generate appropriate responses. Generative AI uses text generation AI (such as LLM) to generate natural conversations. Additionally, the conversation unit guides the user on times for medication, meals, and exercise based on the user's statements.

Step 3: The determination unit determines the user's situation based on the information sent from the AI robot. The determination unit detects the user's body temperature and heart rate with sensors and analyzes the data. The determination unit is a system that includes AI processing or generative AI processing, and it monitors the user's health status and location information. For example, the determination unit analyzes the user's body temperature data and issues an alert if there is an abnormality. It also tracks the user's location information to help reduce missing persons.

The specific processing unit 290 sends the results of the specific processing to the smart device 14. In the smart device 14, the control unit 46A outputs the results of the specific processing to the output device 40. The microphone 38B acquires voice indicating user input in response to the specific processing results. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices. For example, the reception unit is realized by the control unit 46A of the smart device 14 or the specific processing unit 290 of the data processing device 12. For example, the conversation unit is realized by the control unit 46A of the smart device 14 or the specific processing unit 290 of the data processing device 12. For example, the determination unit is realized by the control unit 46A of the smart device 14 or the specific processing unit 290 of the data processing device 12. For example, the guidance unit is realized by the control unit 46A of the smart device 14 or the specific processing unit 290 of the data processing device 12. For example, the output unit is realized by the control unit 46A of the smart device 14 or the specific processing unit 290 of the data processing device 12. For example, the sensor is realized using the camera 42 or microphone 38B of the smart device 14. The correspondence between each unit and the device or control unit is not limited to the examples described above, and various modifications are possible.

### Second Embodiment

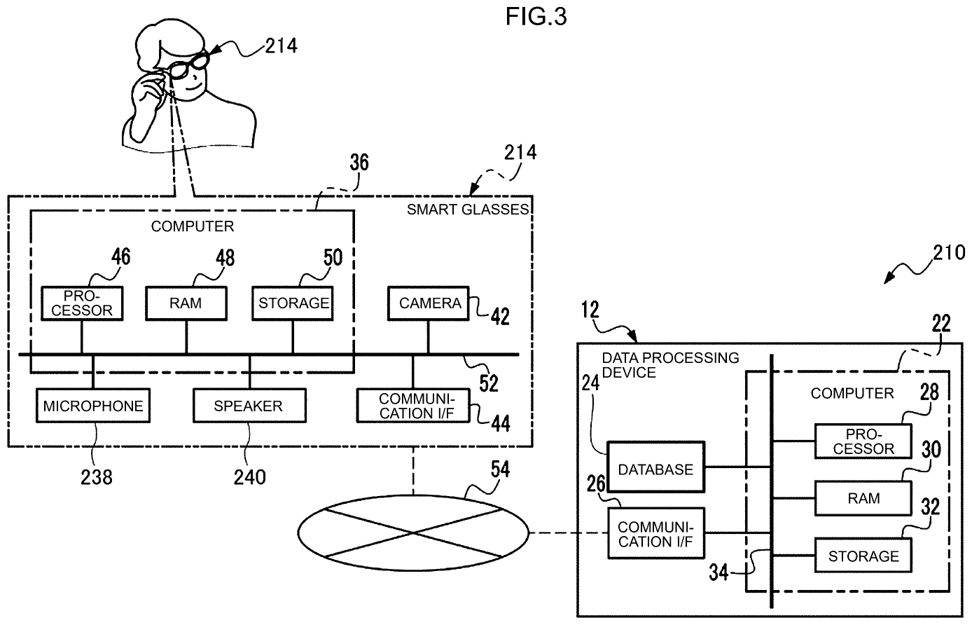

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

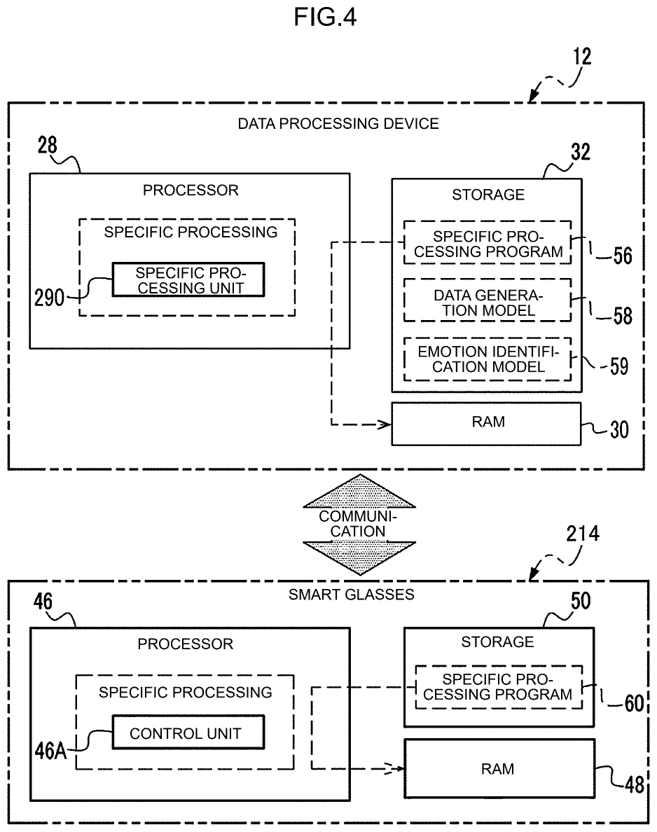

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a Server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

For example, the reception unit is realized by the control unit 46A of the smart glasses 214 or the specific processing unit 290 of the data processing device 12. For example, the conversation unit is realized by the control unit 46A of the smart glasses 214 or the specific processing unit 290 of the data processing device 12. For example, the determination unit is realized by the control unit 46A of the smart glasses 214 or the specific processing unit 290 of the data processing device 12. For example, the guidance unit is realized by the control unit 46A of the smart glasses 214 or the specific processing unit 290 of the data processing device 12. For example, the output unit is realized by the control unit 46A of the smart glasses 214 or the specific processing unit 290 of the data processing device 12. For example, the sensor is realized using the camera 42 or microphone 238 of the smart glasses 214. The correspondence between each unit and the device or control unit is not limited to the examples described above, and various modifications are possible.

### Third Embodiment

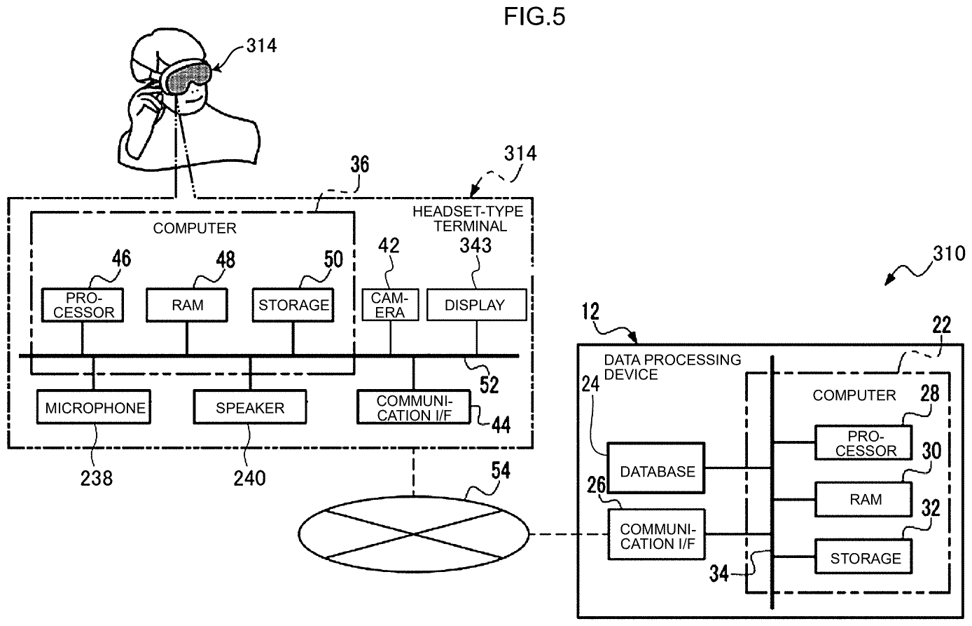

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

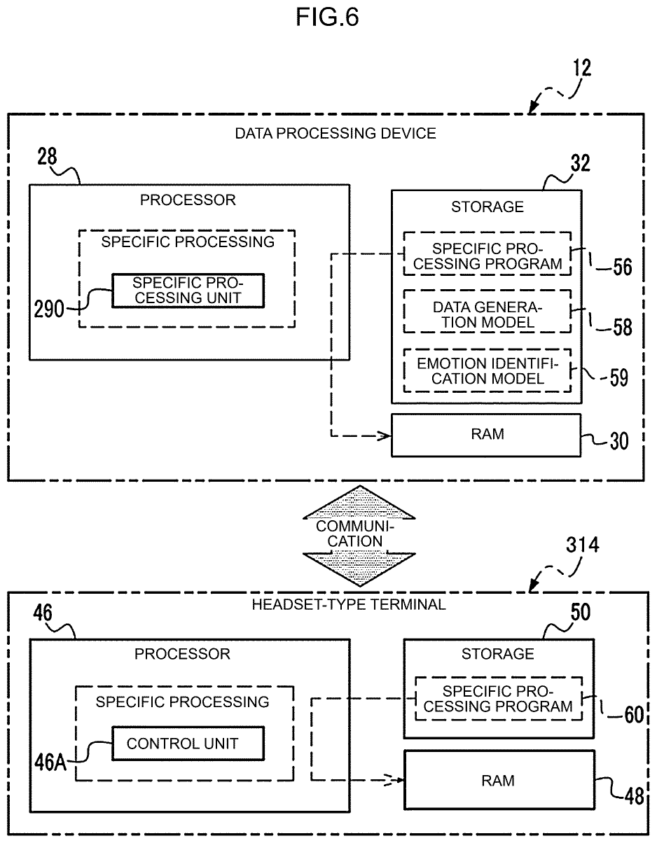

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e. g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

For example, the reception unit is realized by the control unit 46A of the headset-type terminal 314 or the specific processing unit 290 of the data processing device 12. For example, the conversation unit is realized by the control unit 46A of the headset-type terminal 314 or the specific processing unit 290 of the data processing device 12. For example, the determination unit is realized by the control unit 46A of the headset-type terminal 314 or the specific processing unit 290 of the data processing device 12. For example, the guidance unit is realized by the control unit 46A of the headset-type terminal 314 or the specific processing unit 290 of the data processing device 12. For example, the output unit is realized by the control unit 46A of the headset-type terminal 314 or the specific processing unit 290 of the data processing device 12. For example, the sensor is realized using the camera 42 or microphone 238 of the headset-type terminal 314. The correspondence between each unit and the device or control unit is not limited to the examples described above, and various modifications are possible.

### Fourth Embodiment

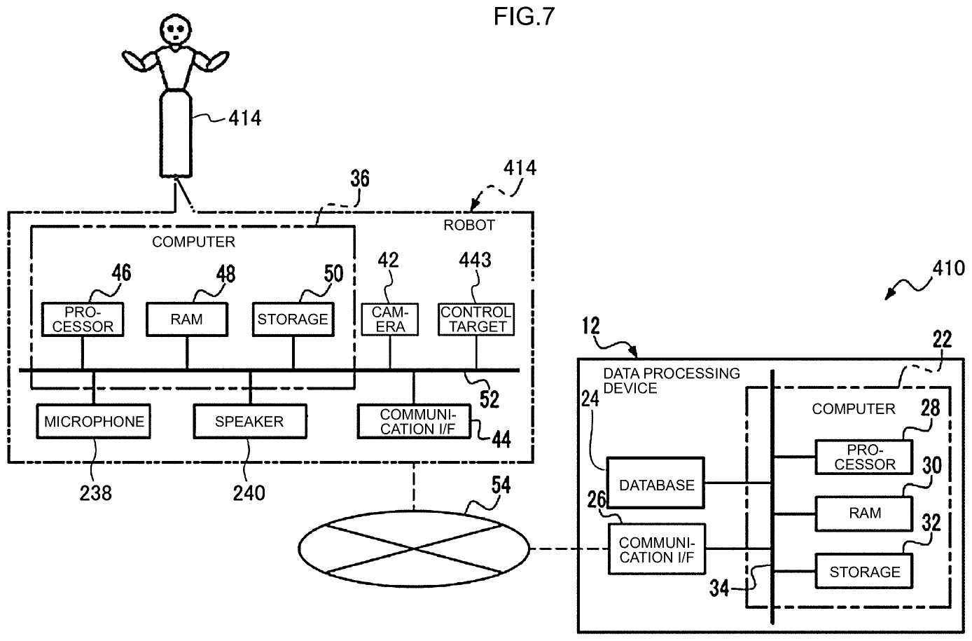

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

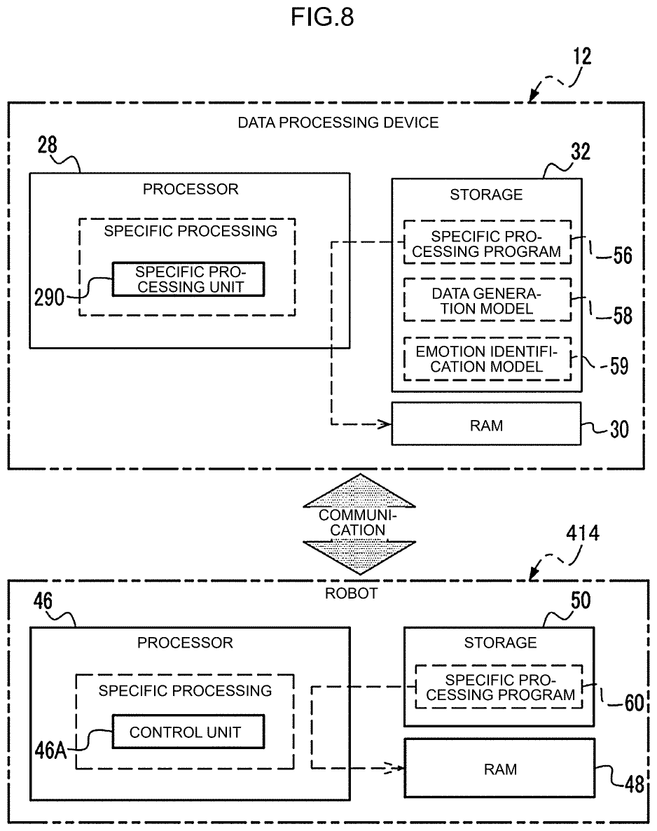

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

For example, the reception unit is realized by the control unit 46A of the robot 414 or the specific processing unit 290 of the data processing device 12. For example, the conversation unit is realized by the control unit 46A of the robot 414 or the specific processing unit 290 of the data processing device 12. For example, the determination unit is realized by the control unit 46A of the robot 414 or the specific processing unit 290 of the data processing device 12. For example, the guidance unit is realized by the control unit 46A of the robot 414 or the specific processing unit 290 of the data processing device 12. For example, the output unit is realized by the control unit 46A of the robot 414 or the specific processing unit 290 of the data processing device 12. For example, the sensor is realized using the camera 42 or microphone 238 of the robot 414. The correspondence between each unit and the device or control unit is not limited to the examples described above, and various modifications are possible.

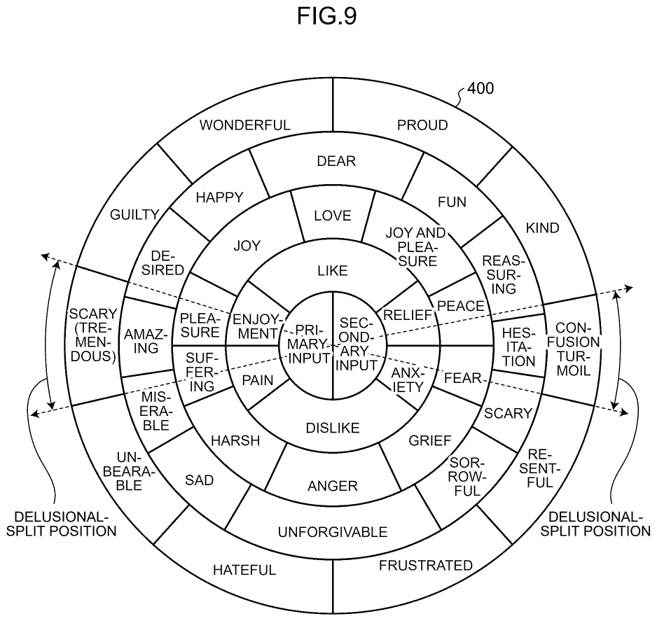

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

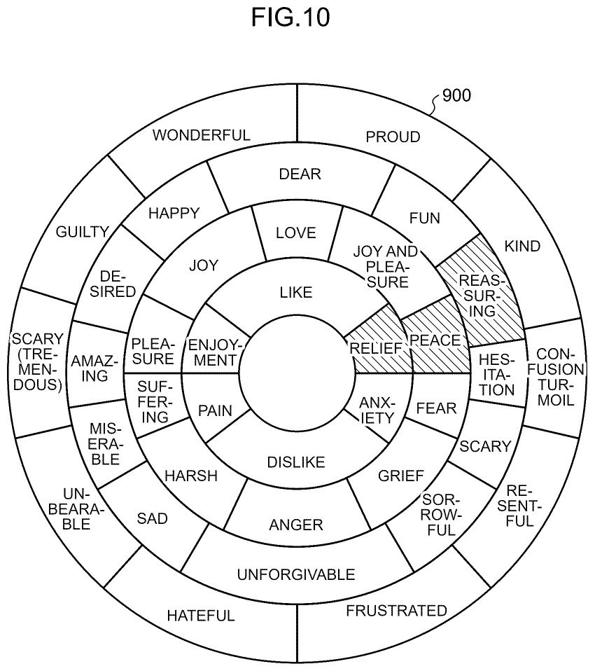

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e. g., a combination of multiple FPGAS or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used. Additionally, the examples described above were explained by dividing into form example 1 and form example 2, but these may be combined.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### [Additional Note 1]

A system including:
a reception unit that accepts user registration;
an AI robot lent to the user whose registration has been accepted by the reception unit; and
a determination unit that determines a user's situation based on the information sent from the AI robot.

### [Additional Note 2]

The system according to Additional Note 1, wherein the AI robot includes:
a conversation unit that engages in conversation with the user;
a guidance unit that specifically guides the user on a time for one of medication, meals, and exercise;
and an output unit that outputs the conversation with the user.

### [Additional Note 3]

The system according to Additional Note 1, further including:
a notification unit that notifies a user's family of determination results by the determination unit.

### [Additional Note 4]

The system according to Additional Note 2, wherein the AI robot includes a sensor that detects user's biometric data, and
the output unit outputs the detected biometric data by the sensor in addition to the conversation with the user.

### [Additional Note 5]

The system according to Additional Note 1, wherein the reception unit estimates user's emotions and simplifies a registration process based on the estimated emotions.

### [Additional Note 6]

The system according to Additional Note 1, wherein the reception unit analyzes a user's past registration history and selects an appropriate registration method.

### [Additional Note 7]

The system according to Additional Note 1, wherein the reception unit filters data based on a user's current health status and a living condition at a time of the registration.

### [Additional Note 8]

The system according to Additional Note 1, wherein the reception unit estimates user's emotions, and determines a priority of registration information based on the estimated emotions.

### [Additional Note 9]

The system according to Additional Note 1, wherein the reception unit prioritizes acquiring highly relevant information by considering user's geographical location information at a time of the registration.

[Additional Note 10] The system according to Additional Note 1, wherein the reception unit analyzes a user's social media activity at a time of the registration, and acquires relevant information.

### [Additional Note 11]

The system according to Additional Note 2, wherein the AI robot estimates user's emotions, and adjusts a robot's action and a conversation content based on the estimated emotions.

### [Additional Note 12]

The system according to Additional Note 2, wherein the AI robot analyzes a user's past behavior pattern during a robot's operation, and selects an appropriate action.

### [Additional Note 13]

The system according to Additional Note 2, wherein the AI robot customizes an action based on a user's current living condition during the robot's operation.

### [Additional Note 14]

The system according to Additional Note 1, wherein the determination unit estimates user's emotions, and adjusts a determination criteria based on the estimated emotions.

### [Additional Note 15]

The system according to Additional Note 1, wherein the determination unit refers to user's past health data during determination to improve accuracy of the determination.

### [Additional Note 16]

The system according to Additional Note 1, wherein the determination unit customizes determination based on a user's current living condition during determination.

### [Additional Note 17]

The system according to Additional Note 2, wherein the conversation unit estimates user's emotions, and adjusts an expression method of the conversation based on the estimated emotion.

### [Additional Note 18]

The system according to Additional Note 2, wherein the conversation unit refers to a user's past conversation history during the conversation to select an appropriate conversation content.

### [Additional Note 19]

The system according to Additional Note 2, wherein the conversation unit customizes a conversation content based on a user's current living condition during the conversation.

### [Additional Note 20]

The system according to Additional Note 2, wherein the conversation unit estimates user's emotions, and adjusts a length of the conversation based on the estimated emotions.

### [Additional Note 21]

The system according to Additional Note 2, wherein the conversation unit determines a priority of the conversation based on a user's submission timing during conversation.

### [Additional Note 22]

The system according to Additional Note 2, wherein based on user's relevance during the conversation, the conversation unit adjusts an order of conversation.

### [Additional Note 23]

The system according to Additional Note 2, wherein the guidance unit estimates user's emotions, and adjusts an expression method of guidance based on the estimated emotions.

### [Additional Note 24]

The system according to Additional Note 2, wherein the guidance unit refers to a user's past behavior history during guidance to select an optimal guidance content.

### [Additional Note 25]

The system according to Additional Note 2, wherein the guidance unit customizes a guidance content based on a user's current living condition during guidance.

### [Additional Note 26]

The system according to Additional Note 2, wherein the guidance unit estimates user's emotions, and adjusts a length of guidance based on the estimated emotions.

### [Additional Note 27]

The system according to Additional Note 2, wherein the guidance unit determines a priority of guidance based on a user's submission timing during guidance.

### [Additional Note 28]

The system according to Additional Note 2, wherein the guidance unit adjusts an order of guidance based on user's relevance during guidance.

### [Additional Note 29]

The system according to Additional Note 2, wherein the output unit estimates user's emotions, and adjusts an expression method of an output based on the estimated emotions.

### [Additional Note 30]

The system according to Additional Note 2, wherein the output unit refers to user's past data during an output to select an optimal output content.

### [Additional Note 31]

The system according to Additional Note 2, wherein the output unit customizes an output content based on a user's current living condition during an output.

### [Additional Note 32]

The system according to Additional Note 2, wherein the output unit estimates user's emotions, and adjusts a length of an output based on the estimated emotions.

### [Additional Note 33]

The system according to Additional Note 2, wherein the output unit determines a priority of an output based on a user's submission timing during an output.

### [Additional Note 34]

The system according to Additional Note 2, wherein the output unit adjusts an order of an output based on user's relevance during an output.

### [Additional Note 35]

The system according to Additional Note 3, wherein the notification unit estimates user's emotions, and adjusts an expression method of a notification based on the estimated emotions.

### [Additional Note 36]

The system according to Additional Note 3, wherein the notification unit refers to user's past data during notification to select an optimal notification content.

### [Additional Note 37]

The system according to Additional Note 3, wherein the notification unit customizes a notification content based on a user's current living condition during notification.

### [Additional Note 38]

The system according to Additional Note 3, wherein the notification unit estimates user's emotions, and adjusts a length of a notification based on the estimated emotions.

### [Additional Note 39]

The system according to Additional Note 3, wherein the notification unit determines a priority of a notification based on a user's submission timing during notification.

### [Additional Note 40]

The system according to Additional Note 4, wherein the sensor estimates user's emotions, and adjusts a sensitivity of the sensor based on the estimated emotions.

### [Additional Note 41]

The system according to Additional Note 4, wherein the sensor refers to user's past data during data acquisition to select an optimal acquisition method.

### [Additional Note 42]

The system according to Additional Note 4, wherein the sensor considers a user's current living condition during data acquisition to select an optimal acquisition method.

### [Additional Note 43]

The system according to Additional Note 4, wherein the sensor estimates user's emotions, and adjusts a frequency of data acquisition based on the estimated emotions.

### [Additional Note 44]

The system according to Additional Note 4, wherein the sensor considers user's geographical location information during data acquisition to select an optimal acquisition method.

1. A robot control system comprising a data processing device and a robot provided to a user, the robot being configured to communicate with the data processing device through a network,
wherein the robot comprises:
an LED provided as an eye of the robot;
a motor configured to drive an arm, a hand, or a foot of the robot;
a sensor set configured to acquire:
biometric data of the user, the biometric data including at least one of heart rate, body temperature, and blood pressure; and
location information of the user;
a first processor configured to control the sensor set, the LED, and the motor; and
a robot communication interface configured to transmit, to the data processing device through the network, the biometric data and the location information, and receive, from the data processing device through the network, information indicating a selected sensor setting and information indicating a selected physical output mode, and
wherein the data processing device comprises:
a storage configured to store registration information of the user, the registration information including a home address of the user and past health data of the user;
a device communication interface configured to communicate with the robot through the network; and
a second processor configured to:
select, based on the past health data stored in the storage, a sensor setting for detection of an abnormality in the user, the sensor setting defining a data acquisition method for the sensor set;
transmit, to the robot through the device communication interface, the information indicating the selected sensor setting;
receive, from the robot through the device communication interface, the biometric data acquired by the sensor set according to the selected sensor setting;
determine a health abnormality of the user by comparing the received biometric data with the past health data stored in the storage;
receive, from the robot through the device communication interface, a plurality of instances of the location information;
determine a wandering condition of the user by determining, based on the plurality of instances of the location information and the home address stored in the storage, that the user has left home and has not returned home for longer than a predetermined time;
select, based on at least one of the determined health abnormality and the determined wandering condition, a physical output mode corresponding to the at least one of the determined health abnormality and the determined wandering condition, the physical output mode identifying at least one of a lighting state of the LED and a motor operation of the motor; and
transmit, to the robot through the device communication interface, the information indicating the selected physical output mode,
wherein the first processor of the robot is further configured to:
control the sensor set based on the information indicating the selected sensor setting; and
based on the information indicating the selected physical output mode, control the LED according to the lighting state or control the motor according to the motor operation to drive the arm, the hand, or the foot of the robot, thereby causing the robot to present, to the user, a user-facing physical alert corresponding to the at least one of the determined health abnormality and the determined wandering condition.

2-9. (canceled)

10. The robot control system of claim 1, wherein the selected sensor setting includes a sensitivity of the sensor set.

11. The robot control system of claim 1, wherein the selected sensor setting includes a frequency at which the sensor set acquires the biometric data.

12. The robot control system of claim 1,
wherein the physical output mode corresponding to the wandering condition identifies the motor operation of the motor, and
wherein the motor operation causes the motor to drive the arm, the hand, or the foot of the robot to perform a gesture corresponding to the wandering condition.

13. The robot control system of claim 1,
wherein the registration information further includes contact information of a family member of the user, and
wherein the second processor is further configured to transmit, using the contact information, a notification corresponding to the health abnormality or the wandering condition.

14. The robot control system of claim 1,
wherein the robot further comprises a speaker, and
wherein the first processor is further configured to control the speaker to output voice guidance corresponding to the health abnormality.

15. The robot control system of claim 1, wherein the robot further comprises a microphone configured to receive a statement of the user.

16. The robot control system of claim 1, wherein the robot has an external appearance of a dog, a cat, or a child.

17. The robot control system of claim 1, wherein the physical output mode identifies both the lighting state of the LED and the motor operation of the motor.

18. The robot control system of claim 1, wherein the second processor is further configured to select the sensor setting based on at least one of a current living condition of the user and the location information.
